(19) United States
(12) Patent Application Publication
(10) Pub. No.: US 2025/0327010 A1
(43) Pub. Date: Oct. 23, 2025

(54) THREE-DIMENSIONAL BIOREACTORS WITH PARTICLE-FACILITATED ANTIBODY COATINGS

(71) Applicant: Southwest Research Institute, San Antonio, TX (US)

(72) Inventors: Jian LING, Spring Branch, TX (US); Madison MORALES, San Antonio, TX (US); Angelica FERNANDEZ, San Antonio, TX (US)

(21) Appl. No.: 18/640,078

(22) Filed: Apr. 19, 2024

(51) Int. Cl. C12M 1/12; C12M 1/00

(52) U.S. Cl. CPC C12M 25/18; C12M 29/06

(57) ABSTRACT

An apparatus and method for T-cell separation, activation, transduction and expansion. Three-dimensional (3D) bioreactors may be employed that include antibody coatings which are preferably antibody coated particles applied to the 3D bioreactor surface. Such 3D bioreactors can be employed for T-cell separation from peripheral blood mononuclear cells including attachment of T-cells to the 3D bioreactor surface for activation and transduction by lentivirus vectors to produce CAR T-cells.

### STATEMENT REGARDING FEDERALLY SPONSORED RESEARCH OR DEVELOPMENT

This invention was made with government support under FDA Contract No. 75F40119C10158. The government has certain rights in the invention.

### FIELD

The present invention relates to an apparatus and method for T-cell separation, activation and transduction. Three-dimensional (3D) bioreactors may be employed that include antibody coatings which are antibody coated particles applied to the 3D bioreactor surface. Such 3D bioreactors can be employed for T-cell separation from peripheral blood mononuclear cells including attachment of T-cells to the 3D bioreactor surface for activation and transduction by lentivirus vectors to produce CAR T-cells.

### BACKGROUND

Chimeric Antigen Receptor (CAR) T-cell therapy is considered a revolutionary approach that relies upon a patient's own immune cells to cure cancer. Five (5) CAR T-cell based therapies have gained FDA approval in the last four years. Numerous clinical trials are in progress. However, consistent, scalable and cost-effective manufacturing for CAR T-cell based therapies remains challenging. Currently, the relatively high cost of CAR T-cell based therapies limits the number of patients who might benefit from this new cancer therapy. The biotechnology industry remains active in its pursuit of new manufacturing platforms and procedures to reduce cost of manufacturing CAR T-cells.

U.S. Pat. No. 10,988,724, entitled Three-Dimensional Bioreactors For Cell Expansion And Related Applications, describes a three-dimensional bioreactor that is composed interconnected voids with a plurality of pore openings between such voids.

U.S. Pat. No. 11,149,244, entitled Three-Dimensional Bioreactor For T-Cell Activation And Expansion For Immunotherapy, describes a method for T-cell expansion that relies upon a 3D bioreactor comprising a plurality of voids having a surface area for cell expansion. A polydopamine coating is applied to the bioreactor surface, a tetrameric protein is then attached to the polydopamine coating, one or more biotinylated antibodies (e.g., anti-CD3 or anti-CD28) are immobilized on the tetrameric protein. One then flows T-cells through the 3D bioreactor having T-cell surface receptors that bind to the biotinylated antibodies and are activated. One then exposes the T-cell to a perfusion media containing oxygen and nutrition to promote T-cell expansion.

U.S. Pat. No. 11,447,731, entitled Three-Dimensional Bioreactors, describes a three-dimensional bioreactor for growth of cells also composed of interconnected voids with a plurality of pore openings between the voids, where the bioreactor is coated with substituted or unsubstituted poly(p-xylylene).

U.S. Publication No. 20210317396 entitled Three-Dimensional Bioreactor For Viral Vector Production describes a three-dimensional bioreactor with a plurality of interconnected voids with a plurality of pore openings between the voids, including seeding the bioreactor with viral vector producing cells and flowing a perfusion medium through the bioreactor and promoting viral vector cell expansion.

U.S. Publication No. 20230139619 entitled Devices For Cell Separation, describes a device for cell separation that includes a plurality of non-random solid geometrical structures and optionally a plurality of non-random solid interconnecting elements between such structures. The surfaces of such device may be coated and functionalized to allow for selective ligand or cell binding and provide a method to separate one or more targeted cells from a plurality of cells.

A need nonetheless remains to provide both methods and devices to improve cellular expansion and in particular T-cell expansion via improved bioreactor designs such as more cost-effective antibody coating on the internal surface of bioreactors.

### SUMMARY

A method for coating a 3D bioreactor with antibody-labelled particles comprising:
a. supplying a 3D bioreactor comprising:
i.a plurality of voids having a diameter D and a plurality of pore openings between said voids having a diameter d, including a void surface area for coating, wherein 90% or more of said voids have a selected void volume (V) that does not vary by more than +/−10.0% and 90% or more of said pore openings between said voids have a value of d that does not vary by more than +/−10.0%; or
ii.a plurality of solid geometrical structures having outer surfaces for coating, wherein 90% or more of said solid geometrical structures have a volume (V) that does not vary by more than +/−10.0%;
b. providing antibody labelled particles comprising an antibody immobilized to a surface of said particles; and
c. coating of said 3D bioreactor void surface area or geometrical structures having outer surfaces with said antibody labelled particles.

A 3D bioreactor comprising:
a. a plurality of voids having a diameter D and a plurality of pore openings between said voids having a diameter d, including a void surface area for coating, wherein 90% or more of said voids have a selected void volume (V) that does not vary by more than +/−10.0% and 90% or more of said pore openings between said voids have a value of d that does not vary by more than +/−10.0%; or
b. a plurality of solid geometrical structures having outer surfaces for coating, wherein 90% or more of said solid geometrical structures have a volume (V) that does not vary by more than +/−10.0%; and
c. a coating of antibody labelled particles on said 3D bioreactor void surface area or on said outer surfaces of said geometrical outer surfaces.

### BRIEF DESCRIPTION OF THE DRAWINGS

FIG. 1A illustrates a portion of a 3D bioreactor herein having a plurality of non-random voids and non-random pore openings.

FIG. 1B illustrates, in cross-section, a non-random void.

FIG. 2A illustrates a portion of a 3D bioreactor herein having non-random solid geometrical structures and optional solid non-random interconnecting elements.

FIG. 2B also illustrates a portion of a 3D bioreactor herein having non-random solid geometrical structures and optional solid non-random interconnecting elements.

FIG. 2C provides yet another illustration of a portion of a 3D bioreactor herein having non-random solid geometrical structures and optional solid non-random interconnecting elements.

FIG. 3 illustrates a portion of a 3D bioreactor herein showing the exemplary use of non-random spheres and non-random interconnecting elements.

FIG. 4 illustrates the bioreactor of either the first or second configuration positioned in a housing between an inlet and outlet for fluid flow.

FIG. 5 illustrates a portion of the 3D bioreactor surface herein within the non-random voids shown in FIG. 1 or on the surface of the non-random geometrical structures shown in FIG. 2A where the surface includes the immobilized particle-facilitated antibody coating.

FIG. 6A illustrates the 3D bioreactor as shown in FIG. 1 or FIG. 2A positioned within a housing for inflow of a mixed population of T-cells, B-cells, monocytes and NK cells.

FIG. 6B illustrates the placement of the bound/activated T-cells in the 3D bioreactor 34.

### DETAILED DESCRIPTION

The present invention is directed at three-dimensional (3D) bioreactors that are coated with functionalized particles. Namely, antibody-labelled particles are coated onto the 3D bioreactor surface. The preferred 3D bioreactor surface herein for such antibody-labelled particle coating is preferably of two types, namely either a “positive” or “negative” 3D bioreactor, as further described herein.

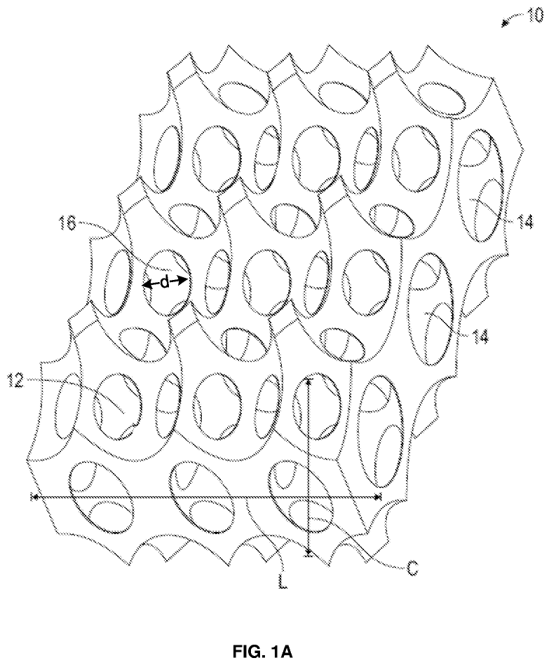

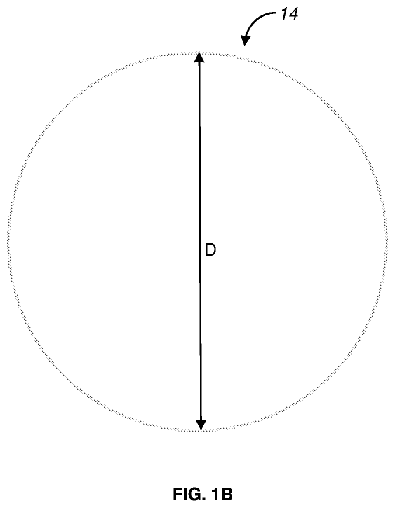

The first type of 3D reactor 10 for coating with antibody-labelled particles, is illustrated in FIG. 1A. The 3D bioreactor shown in cut-away view includes a continuous interconnected void surface area 12 that includes a plurality of interconnected non-random voids 14 which are preferably of spherical shape with internal concave surfaces. By reference to non-random it be understood that one can now identify a targeted or selected number of voids in the 3D bioreactor that results in an actual repeating void size and/or geometry of a desired tolerance. The 3D bioreactor also includes non-random interconnected pore openings 16 as between the voids, which pore openings may also be of a desired tolerance. The 3D bioreactor also ultimately defines a layer of non-random voids (see arrow “L”), and the multiple layers of the 3D bioreactor may then allow for identification of such non-random voids within a column (see arrow “C”). It is also useful to now note that such 3D bioreactor may be termed a “negative” bioreactor, since as noted, it relies about a plurality of interconnected non-random voids and non-random interconnected pore openings.

The 3D bioreactor in FIG. 1A is preferably such that the plurality of voids 14 have a diameter D (the longest distance between any two points on the internal void surface), the plurality of pore openings have a diameter d (the longest distance between any two points at the pore opening), where D>d. See FIG. 1B wherein the pore openings are not shown. In addition, 90.0% or more of the voids, or even 95.0% or more of such voids, or even 99.0% to 100% of such voids have a void volume (V) whose tolerance is such that it does not vary by more than +/−10.0%, or +/−5.0%, or +/−2.5%, or +/−1.0%, or +/−0.5%, or +/−0.1%.

Furthermore, 90.0% or more of the pore openings, or even 95.0% or more of the pore openings, or even 99.0% to 100% of the pore openings between the voids, indicate a value of d whose tolerance does not vary by more than +/−10.0%, or +/−5.0%, or +/−2.5%, or +/−1.0%, or +/−0.5%, or +/−0.1%. The diameter (D) of the voids preferably have a value in the range of 0.09 mm to 100.0 mm, including all individual values and increments therein. For example, the diameter of the voids may be in the range of 0.2 mm to 50.0 mm, or 0.2 mm to 50.0 mm, or 0.4 mm to 50.0 mm, or 0.4 mm to 25.0 mm. The diameter of the pore openings (d) is preferably in the range of 0.01 mm to 10.0 mm, including all values and increments therein. For example, the pore opening diameter (d) may be in the range of 0.05 mm to 2.0 mm or 0.1 mm to 2.0 mm.

The 3D bioreactor herein may also preferably be constructed in a second configuration comprising a plurality of non-random solid geometrical structures and optionally a plurality of interconnecting elements. This second configuration may therefore be understood as a “positive” 3D bioreactor. The solid geometrical structure may preferably include spheres, ovals, and/or polygonal shapes, thereby presenting an outer surface for production of CAR T-cells herein. As noted, such solid geometrical structures may optionally be connected via a plurality of solid interconnecting elements that may also assume a variety of geometrical shapes, including rod or columnar shape, oval shape, and/or polygonal type shape. Such solid interconnecting structure may also provide an outer surface for production of CAR T-cells. Both the solid geometrical structures and/or the solid interconnecting elements themselves are not necessarily completely solid and may contain partially hollow interiors to place nutrients and/or other reagents to improve the performance of such 3D bioreactor for production of CAR T-cells.

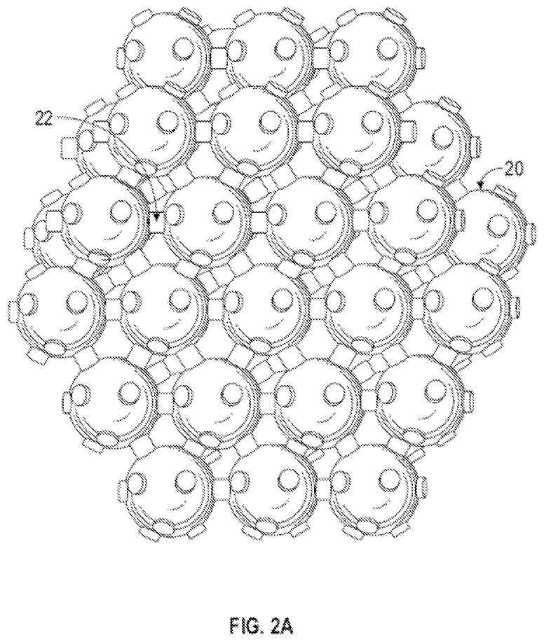

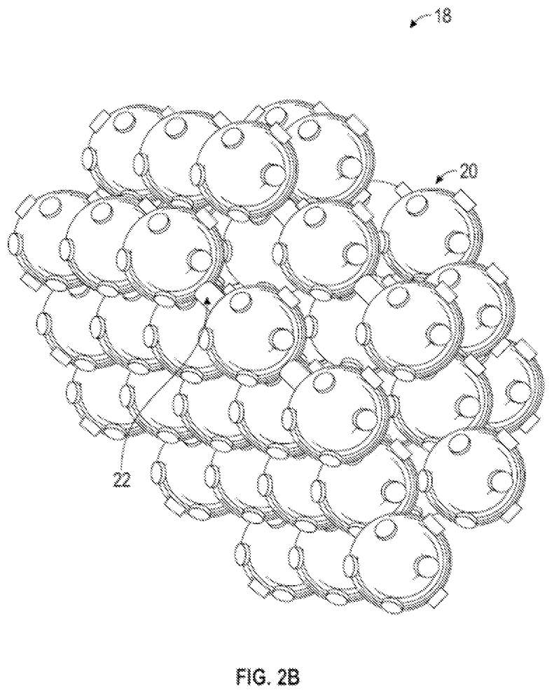

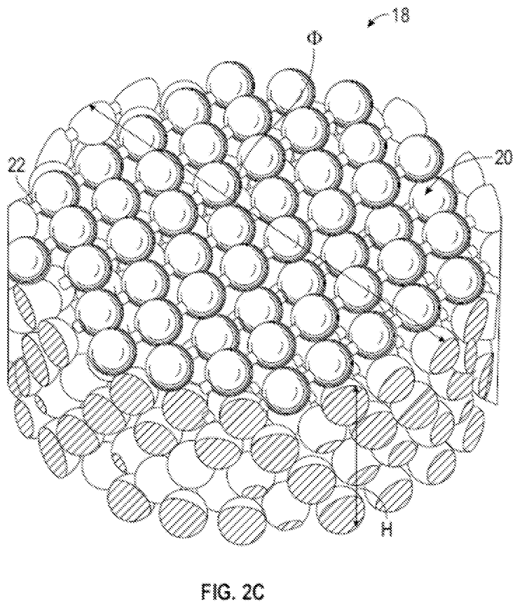

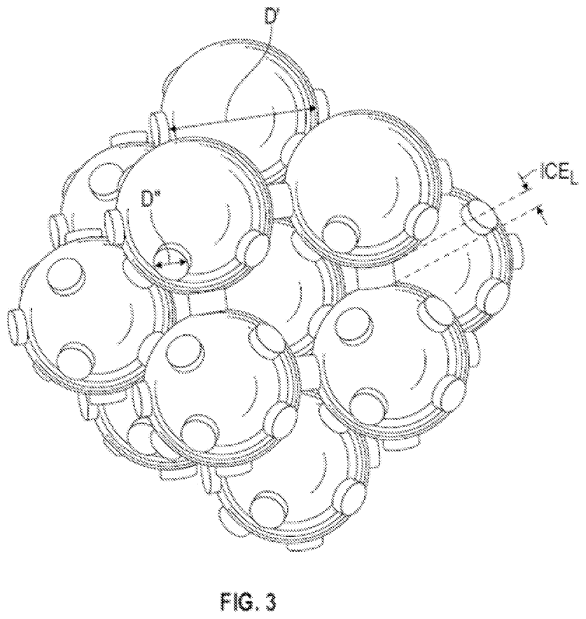

FIGS. 2A and 2B illustrate the second configuration of a 3D bioreactor 18 herein where the solid non-random geometrical structures and optional solid non-random interconnecting elements preferably comprise spheres 20 and interconnecting rods 22. As illustrated in FIG. 2C, the 3D bioreactor 18 preferably has a diameter Φ in the range of 2.0 mm to 10,000 mm and a height H in the range of 1.0 mm to 5,000 mm. Preferably, the 3D bioreactor 18 has Φ/H in the range of greater than 1:1. A portion of the 3D bioreactor 18 is illustrated in FIG. 3 with respect to the exemplary use of spheres and the optional use of interconnecting rod elements. The non-random solid geometrical shapes herein preferably have a diameter D′ (the longest distance between two points on the outer surface of the solid geometrical structure and through the structure interior) in the range of 2.0 μm to 25.0 mm, including all values and increments therein. Accordingly, D′ may have a value in the range of 200 μm to 25.0 mm, or 5.0 μm to 10.0 mm, or 5.0 μm to 6.0 mm, or 1.0 mm to 25.0 mm. The solid geometrical interconnecting elements (ICE) preferably have a diameter D″ in the range of 1.0 μm to 12.5 mm, including all values and increments therein. Accordingly, D″ may preferably have a value of 1.0 μm to 3.0 mm. The length of the solid interconnecting elements (ICE_{L}) preferably has a value of 0.1 μm to 25.0 mm, including all values and increments therein. Accordingly, the ICE_{L} may have a value of 100.0 μm to 5.0 mm, or 100.0 μm to 3.0 mm. It is preferred that the diameter of the solid interconnecting structures (e.g., rods) are less than half of the value of the diameter of the solid geometrical shapes (e.g., spheres).

Similar to the first configuration of the 3D bioreactor, the second configuration can also be characterized by its overall non-random characteristics. That is, with respect to the solid geometrical structures (e.g., spheres 20), 90% or more of such solid geometrical structures, or even 95.0% or more of such solid geometrical structures, or even 99.0% to 100% of such solid geometrical structures, define a volume whose tolerance is such that it does not vary by more than +/−10.0%, or +/−5.0%, or +/−2.5% or +/−1.0% or +/−0.5% or +/0.1%. Similarly, with respect to the optional use of the solid interconnecting elements (e.g., rods 20), 90% or more of such solid interconnecting elements, or even 95.0% or more of such solid interconnecting elements, or even 99.0% to 100% of such solid interconnecting elements, define a volume whose tolerance is such that it does not vary by more than +/−10.0%, or +/−5.0%, or +/−2.5% or +/−1.0% or +/−0.5% or +/−0.1%.

The 3D bioreactor device of the first configuration or second configuration is preferably made of biocompatible or bio-inert polymeric materials such as polystyrene, polycarbonate, acrylonitrile-butadiene-styrene (ABS), polylactic acid (PLA), polycaprolactone (PCL) used in FDM (fused deposition modeling) 3D printing technology. Reference to biocompatible or bio-inert should be understood as a material that is non-toxic to the culturing cells. In addition, the polymeric materials for the device of the first or second configuration are preferably selected from those polymers that at not susceptible to hydrolysis during cell cultivation, such that the amount of hydrolysis does not exceed 5.0% by weight of the polymeric material present, more preferably it does not exceed 2.5% by weight, and most preferably does not exceed 1.0% by weight. The device of the first or second configuration may also be made of biocompatible photosensitive materials (e.g., Pro3Dure, Somos WaterShed XC 11122, etc.) used in SLA (stereolithography) and DLP (digital light processing) 3D printing technologies. Furthermore, the device of the first or second configuration may be formed of an interpenetrating polymer network (IPN). An IPN is reference to a polymer comprising two or more networks which are at least partially interlaced on a polymer scale but not covalently bonded to each other.

The 3D bioreactor devices of the first or second configuration herein are also preferably formed from material that indicates a Shore D Hardness of at least 10, or in the range of 10-95, and more preferably in the range of 45-95. In such regard, it is also worth noting that the devices herein preferably do not make use of a hydrogel type structure, which may be understood as a hydrophilic type polymeric structure, that includes some amount of crosslinking, and which absorbs significant amounts of water (e.g., 10-40% by weight). It is also worth noting that the devices herein preferably do not make use of collagen, alginate, fibrin and other polymers that cells can easily be digested and undergo remodeling.

Furthermore, the 3D bioreactor devices herein of the first or second configuration are preferably made from materials that have a Tensile Modulus of at least 0.01 GPa. More preferably, the Tensile Modulus has a value that is in the range of 0.01 GPa to 20.0 GPa, at 0.01 GPa increments. Even more preferably, the Tensile Modulus for the material for devices herein are in the range of 0.01 GPa to 10.0 GPa or 1.0 GPa to 10 GPa. For example, with respect to the earlier referenced polymeric materials suitable for manufacture of the devices herein, polystyrene indicates a Tensile Modulus of about 3.0 GPa, polycarbonate at about 2.6 GPa, ABS at about 2.3 GPa, PLA at about 3.5 GPa and PCL at about 1.2 GPa.

The 3D bioreactor devices herein of either the first or second configuration with such preferred regular geometric characteristics and/or surface area are preferably fabricated by additive manufacturing technologies, such as fused deposition modeling FDM, selective laser sintering (SLS), stereolithography (SLA), digital light processing (DLP) 3D printing technologies, etc., according to computer generated designs made available by, e.g., a SolidWorks™ computer-aided design (CAD) program.

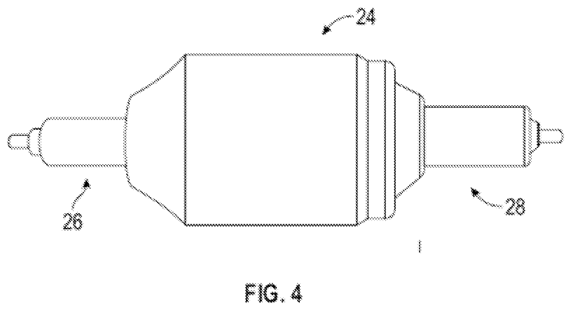

The 3D bioreactor devices of the first or second configuration may then be configured such that they amount to a fixed bed along with an inlet and outlet to allow for inflow and outflow of fluid. Reference is made to FIG. 4 wherein the 3D bioreactor device of either the first or second configuration noted above may be positioned in a housing 24 and then placed between and inlet 26 and outlet 28 for which inflow and outflow of fluid may be provided containing cells for separation.

With regards to optional use of surface coating of the 3D bioreactor devices of either the first or second configuration, preferably, such coatings comprise substituted or unsubstituted poly(p-xylylene) from the polymerization of parylene monomers, β-casein or polydopamine (PDA). Such coatings may preferably be present at a thickness in the range of 200 Angstroms to 100.0 μm.

Accordingly, the coating procedure preferably relies upon the use of parylene monomers, e.g., [2.2]paracyclophanes, that may be preferably functionalized with identified R₁, R₂, R₃ and R₄ groups according to the following general reaction scheme. It should be appreciated that in the scheme below, the start of polymerization is initiated by a ring opening at elevated temperature (˜550° C.) in the low pressure gas phase remotely prior to deposition on the 3D device which is preferably maintained at relatively lower temperature (e.g., ≤100° C.):

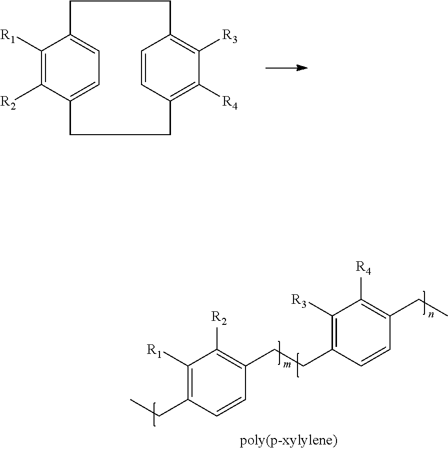

In the above, when one of the R groups per repeat unit “m” and/or repeat unit “n” is chlorine, and the other R groups are hydrogen, the above represents the polymerization of parylene C. It is a USP Class VI and ISO-10993-6 certified biocompatible material. The values of “m” and “n” of the identified crosslinked, repeating units are such that molecular weight values are relatively high, such as ˜500,000. It is therefore contemplated that the use of the parylene monomers and ensuing polymeric coatings are such that one may now coat the devices of the above reference first or second configuration herein with an impermeable film. The film may preferably have a thickness between 200 Angstroms to 100.0 μm. It may be appreciated that R₁, R₂, R₃, and R₄ may be selected from hydrogen, a halogen (—Cl or —Br) as well as other functional groups such as amines (—NH₂), aliphatic aldehydes (—CHO), carboxylic acid functionality (—COOH), hydroxyl (—OH) or carboxylate functionality as in —C(O)CF₃. One may also initially coat with a first layer of impermeable parylene C followed by a coating of a different parylene, e.g., wherein R₁, R₂, R₃, and R₄ may then be selected from an amines (—NH₂) and/or aldehyde (—CHO) functionality. Accordingly, one may provide polymeric coatings for the devices herein of the first and/or second configuration, wherein the coating comprises a plurality of layers, each with its own particular and different chemical composition (i.e. the identity of at least one of R₁, R₂, R₃, and R₄ are different between at least two of the layers).

One preferred method of coating the surface of the devices herein with functionalized poly(p-xylylene) applies when one or more of the R₁, R₂, R₃ and/or R₄ groups noted above comprise ester carboxylic acid functionality. In such a case, one may utilize N-hydroxysuccinimide (NHS) to form an ester linkage. Next, NH₂-mPEG (methoxy terminated oligoethylene glycol) or NH₂-PEG-biotin may be covalently bonded to the device surface via the amine-NHS ester reaction to form an amide bond. Then, avidin or NeutrAvidin™ (deglycosylated native avidin protein) or streptavidin can be bound to the biotin. As avidin/deglycosylated native avidin protein/streptavidin have four bonding sites, the remaining three sites are then available to bind biotinylated antibodies, such as anti-CD3 and anti-CD28 to capture T cells through surface receptors specific to these antibodies.

Coating of the 3D Bioreactors with Distributed Antibody Clusters Facilitated by Particles

The 3D bioreactors herein may also more preferably be coated with antibody labelled particles on the 3D bioreactor surfaces. As noted, one may place antibody coatings directly on the internal surface of 3D bioreactors. However, due to the relative high cost of antibodies, the direct coating of antibodies onto the 3D bioreactor surface may not be as cost-effective as compared to the alternative antibody coated beads, such as antibody coating of Dynabeads™ spherical particles (ThermoFisher), which offer higher relative surface to volume ratios. For example, 1.8 ml of phosphate buffered saline (PBS) solution, containing 10 μg/ml of anti-CD3 antibody, is required to coat a 3D bioreactor having a surface area of 25 cm2 containing a plurality of interconnected non-random voids with a plurality of non-random pore openings between the voids. However, to coat 3.9×107 Dynabeads™ (spherical superparamagnetic particles having a 4.5 μm diameter), having an equivalent total surface area of 25 cm2, the coating can be carried out in 0.1 ml of PBS (phosphate buffer solution) with 10 μg/ml of anti-CD3 antibody. As can be seen, the latter would only need about 1 μg of antibody instead of 18 μg in the former case.

In order to improve the coating efficiency of the 3D bioreactors, and preferably for cases where a continuous surface coatings of antibodies are not necessary, particle-facilitated antibody coatings are now employed on the 3D bioreactor surface. The particles preferably include any biocompatible particle that can serve to immobilize an antibody. Accordingly, the particles may comprise silica (SiO₂) particles and/or biocompatible polymeric particles. Antibody labelled particles herein are therefore a reference to particles that have an antibody first immobilized to the particle, which may preferably occur by covalent attachment or relatively strong non-covalent binding, e.g., biotin tetrameric protein binding. The diameter of particles can range between 10 nm and 10 μm, including all individual values and increments therein. More preferably the particles have a diameter of less than or equal to 1.0 μm. Accordingly, the particles also preferably have a diameter in the range of 100 nm to 1000 nm (1.0 μm), including all individual values and increments therein.

The particles may therefore be preferably coated with a biotin binding molecule, such as a tetrameric protein (protein with quaternary structure), which includes avidin, streptavidin or deglycosylated native avidin protein. The deglycosylated native avidin protein is sold under the name NeutraAvidin™. The particles with a biotin binding molecule may then be coated with biotinylated antibodies. More specifically, one can now immobilize biotinylated antibodies such as anti-CD3, anti-CD19, anti-CD22, anti-CD25 and anti-CD28 to the particle surface that has been coated with a biotin binding molecule via, e.g., the biotin-avidin/streptavidin/NeutrAvidin™ binding mechanism. A biotinylated antibody is reference to the covalent attachment of any one or more of the identified antibodies to the biotin binding molecule now associated with the particle surface.

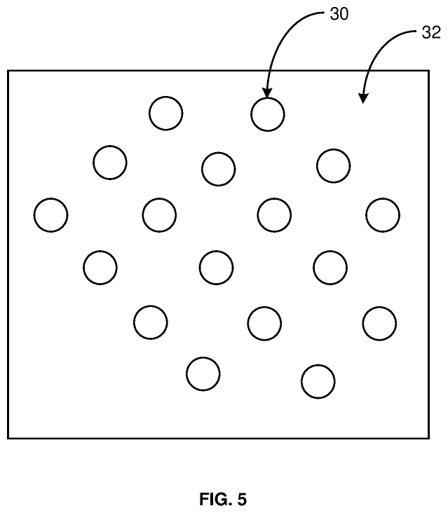

The antibody labelled particles now described herein provide relatively easy convection of fluid suspended particles flowing into the 3D bioreactors herein with subsequent attachment to the surfaces of the 3D bioreactors. Such attachment to the 3D bioreactor surface is contemplated to occur through a region of antibodies on the particles that are adsorbed to the hydrophobic 3D bioreactor surface via a hydrophobic-hydrophobic interaction. The immobilized antibody-labelled particles preferably provide an array of discrete antibody clusters that face out of the 3D bioreactor surface. FIG. 5 illustrates a portion of the 3D bioreactor surface herein, wherein by controlling the number of antibody labelled particles 30 on the surface 32 of the 3D bioreactor, the average surface density of the antibody labelled particles can be determined for a relatively uniform coating. The 3D bioreactor surface in FIG. 5 may be a portion of the 3D bioreactor surface within the non-random voids 14 shown in FIG. 1A or on the surface of the non-random geometrical structures 20 (e.g., spheres) or non-random interconnecting elements (e.g. rods) shown in FIG. 2A. The average surface density of the antibody labelled particles on the surface of the 3D bioreactor preferably falls in the range of 3.9×105 particles/cm² to 3.2×10⁷ particles/cm². The average distance between the antibody labelled particles on the 3D bioreactor surface preferably falls in the range of 1.0 μm to 9.0 μm.

It may therefore be appreciated that the 3D bioreactors herein may be directly coated with discrete antibody arrays facilitated by particles. Or, the 3D bioreactors herein may first be coated with poly(p-xylylene), or β-casein or polydopamine (PDA) and then coated with discrete antibody arrays facilitated by particles.

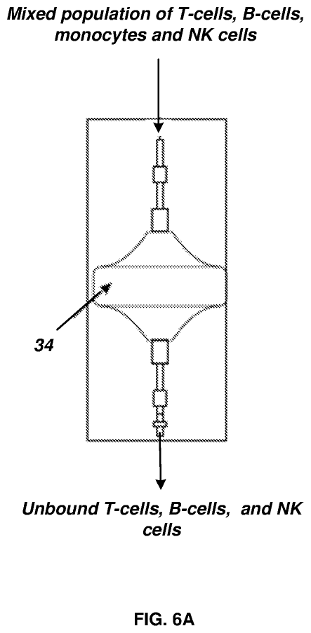

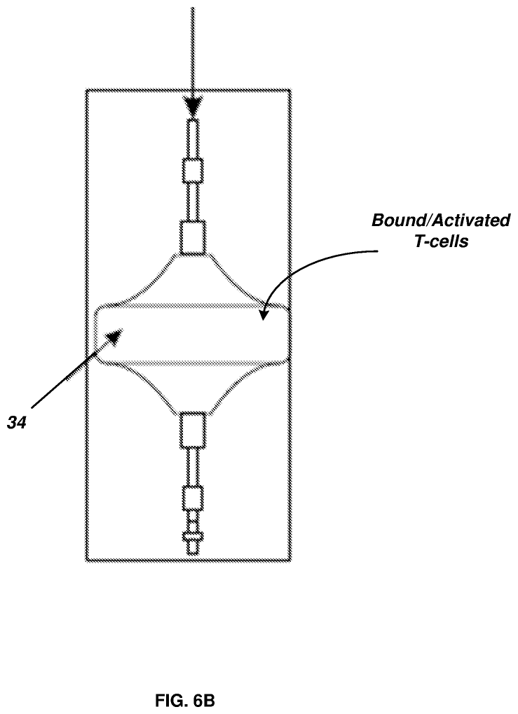

Attention is directed to FIG. 6A where a 3D bioreactor 34 (as shown in FIG. 1A or FIG. 2A) coated with discrete antibody arrays facilitated by particles is positioned within a housing for inflow and outflow of fluid so that it may receive fluid containing peripheral blood mononuclear cells (PBMCs), or a mixed population of T-cells, B-cells, monocytes and NK cells. More specifically, a mixed population of lymphocytes 70-90% (T-cells, B-cells, NK cells), 10-20% monocytes and 1-2% dendritic cells. The frequencies of cell types within the lymphocyte population include 70-85% CD3+ T-cells, 5-10% B-cells, and 5-20% NK cells. The CD3+ lymphocytes are composed of CD4+, CD8+ T-cells, in about a 2:1 ratio. The cell surface receptors on T-cells (e.g. CD3+, CD28+ coactivator receptor) can bind to the antibodies (e.g., anti-CD3, anti-CD28) immobilized on the 3D bioreactor surface via the particles. FIG. 6B shows the bound/activated T-cells on the internal surface of the 3D bioreactor 34.

### WORKING EXAMPLES

### Working Example 1—Coating of 3D Bioreactor with Antibody Labelled Silica Particles

Silica particles having a 1.0 μm diameter were coated with streptavidin and then labeled with biotinylated anti-CD19, anti-CD22 and anti-CD-25 antibodies. About 9.2×10⁸ antibody coated particles were suspended in 2.0 ml of a phosphate buffer saline (PBS) solution and circulated through a 3D bioreactor as illustrated and described in FIGS. 2A-2C and 3, having a internal surface area of 100 cm². The circulation took place for about 16 hours at 2° C. to 8° C. The flow rate was 0.1 mL/min. After coating, the unbound particles were flushed out by 10 ml of PBS flowing from the 3D bioreactor inlet to outlet at the rate of 1 mL/min. At the same flow rate, a second 10 mL of PBS was flushed through the 3D bioreactor from outlet to inlet. The unbound particles flowing out of the reactor were collected and counted to determine the number of antibody coated particles remaining in the 3D bioreactor. Table 1 below shows that an average of 98% of the antibody coated silica particles remained attached to the 3D bioreactor surface.

TABLE 1
Immobilization of Antibody Labelled 1.0 μm Silica
Particles on 3D Bioreactor Surface

| | Particles | Flushed Out | Percent Adhered To |
|---|---|---|---|
| | Used In | Nonbound | 3D Bioreactor |
| Trial | Coating | Particles | Surface |
| 1 | 9.20E+08 | 3.47E+06 | 99.62 |
| 2 | 9.20E+08 | 6.00 + 06 | 99.35 |
| 3 | 9.20E+08 | 3.18 + 07 | 97.38 |
| 4 | 9.20E+08 | 5.98E+06 | 99.49 |
| Average | 9.20E+08 | 1.18E+07 | 98.96 |
| STD | 1.00E+05 | 1.34E+07 | 1.06 |

### Working Example 2—Coating of 3D Bioreactor with Antibody Labelled Silica Particles

A 3D bioreactor as illustrated and described in FIG. 1 having an internal surface area of 250 cm², and an internal fluid volume of about 13 mL was also coated with antibody coated silica particles. Namely, about 4.56×10⁹ streptavidin coated 1.0 μm silica particles (Bands Lab, Cat #CS1001) were added to a 1.5 ml Eppendorf tube. The particles were washed with sterilized PBS twice to remove any preservation agent such as sodium azide (NaN₃). After washing, the particles were first coated with 32 μg/mL of biotinylated sheep anti-mouse secondary antibody and the further coated with 15 μg/mL of mouse IgG anti-CD3 and 15 μg/mL of mouse IgG anti-CD28 antibodies. All coatings were carried out in the 1.5 mL Eppendorf tube with 500 μL of coating solution on a shake at room temperature for 2 hours.

The antibody-coated silica particles were re-suspended in 14 mL of PBS which was circulated through the 3D bioreactor at a flow rate of 0.1 mL/min. The perfusion-based particle coating was carried out for about 16 hours at 2° C. to 8° C. The perfusion flow direction is changed at 8 hours. After coating, a total of about 140 mL of PBS, or 10 times the 3D bioreactor internal void volume was flushed through the 3D bioreactor by gravity, 70 mL of PBS in each direction, to eliminate non-bound particles. Typically, about 3×10⁹ particles were attached to the 3D bioreactor's 250 cm² internal surface, generating an average 1.6 μm distance between the 1.0 μm diameter antibody labelled silica particles on the 3D bioreactor surface.

What is claimed is:

1. A method for coating a 3D bioreactor with antibody-labelled particles comprising:
a. supplying a 3D bioreactor comprising:
i. a plurality of voids having a diameter D and a plurality of pore openings between said voids having a diameter d, including a void surface area for coating, wherein 90% or more of said voids have a selected void volume (V) that does not vary by more than +/−10.0% and 90% or more of said pore openings between said voids have a value of d that does not vary by more than +/−10.0%; or
ii. a plurality of solid geometrical structures having outer surfaces for coating, wherein 90% or more of said solid geometrical structures have a volume (V) that does not vary by more than +/−10.0%;
b. providing antibody labelled particles comprising an antibody immobilized to a surface of said particles; and
c. coating of said 3D bioreactor void surface area or geometrical structures having outer surfaces with said antibody labelled particles.

2. The method of claim 1 wherein said 3D bioreactor comprises biocompatible material.

3. The method of claim 1 wherein said void surface area or said geometrical structure outer surfaces are initially coated with substituted or unsubstituted poly(p-xylylene), β-casein or polydopamine.

4. The method of claim 1 wherein said antibody labelled particles comprise biocompatible particles.

5. The method of claim 1 wherein said antibody labelled particles have a particle diameter of 10 nm to 10.0 μm.

6. The method of claim 1 wherein said antibody labelled particles comprise silica particles.

7. The method of claim 1 wherein said antibody labelled particles comprise polymeric particles.

8. The method of claim 1 wherein said antibody labelled particles comprise particles coated with a biotin binding molecule wherein said biotin binding molecule is coated with biotinylated antibodies.

9. The method of claim 8 wherein said biotin binding molecule comprises a tetrameric protein.

10. The method of claim 9 wherein said tetrameric protein comprises avidin, streptavidin or de-glycosylated native avidin protein.

11. The method of claim 8 wherein said biotinylated antibodies are selected from the group consisting of anti-CD3 antibody, anti-CD22 antibody, anti-CD25 antibody and anti-CD28 antibody.

12. The method of claim 8 further including flowing T-cells through said 3D bioreactor, wherein said T-cells bind to said biotinylated antibodies which T-cells are activated and transduced to CAR T-cells.

13. The method of claim 10 wherein said T-cells are transduced with lentivirus vectors.

14. A 3D bioreactor comprising:
a. a plurality of voids having a diameter D and a plurality of pore openings between said voids having a diameter d, including a void surface area for coating, wherein 90% or more of said voids have a selected void volume (V) that does not vary by more than +/−10.0% and 90% or more of said pore openings between said voids have a value of d that does not vary by more than +/−10.0%; or
b. a plurality of solid geometrical structures having outer surfaces for coating, wherein 90% or more of said solid geometrical structures have a volume (V) that does not vary by more than +/−10.0%; and
c. a coating of antibody labelled particles on said 3D bioreactor void surface area or on said outer surfaces of said geometrical outer surfaces.

15. The 3D bioreactor of claim 14 wherein said 3D bioreactor comprises biocompatible material.

16. The 3D bioreactor of claim 14 wherein said void surface area or said geometrical structure outer surfaces are initially coated with substituted or unsubstituted poly(p-xylylene), β-casein or polydopamine.

17. The 3D bioreactor of claim 14 wherein said antibody labelled particles comprise biocompatible particles.

18. The 3D bioreactor of claim 14 wherein said antibody labelled particles comprise silica particles.

19. The 3D bioreactor of claim 14 wherein said antibody labelled particles comprise polymeric particles.

20. The 3D bioreactor of claim 14 wherein said antibody labelled particles have a particle diameter of 10 nm to 10.0 μm.

21. The 3D bioreactor of claim 14 wherein said antibody labelled particles comprise particles coated with a biotin binding molecule wherein said biotin binding molecule is coated with biotinylated antibodies.

22. The 3D bioreactor of claim 21 wherein said biotin molecule comprises a tetrameric protein.

23. The 3D bioreactor of claim 21 wherein said biotinylated antibodies are selected from the group consisting of anti-CD3 antibody, anti-CD22 antibody, anti-CD25 antibody and anti-CD28 antibody.
